(19) United States
(12) Patent Application Publication
(10) Pub. No.: US 2022/0047514 A1
(43) Pub. Date: Feb. 17, 2022

(54) SOLID ORAL PHARMACEUTICAL COMPOSITIONS FOR CHRONOTROPIC ADMINISTRATION OF DIPEPTIDYL PEPTIDASE IV INHIBITORS

(71) Applicant: DPL PHARMA S.P.A., Rozzano (MI) (IT)

(72) Inventors: Massimo Pedrani, Melide (CH); Chiara Conti, Vigolo Marchese (PC) (IT); Salvatore Agostino Giammillari, Rozzano (MI) (IT); Giuseppe Maccari, Voghera (PV) (IT)

(73) Assignee: DPL PHARMA S.P.A., Rozzano (MI) (IT)

(21) Appl. No.: 17/312,974

(22) PCT Filed: Dec. 12, 2019
(86) PCT No.: PCT/IB2019/060688

§ 371 Date: Jun. 11, 2021

(30) Foreign Application Priority Data
Dec. 14, 2018 (IT) 102018000011119

(51) Int. Cl. A61K 9/28; A61K 31/4985; A61K 9/00

(52) U.S. Cl. CPC A61K 9/2866; A61K 9/2846; A61K 9/0053; A61K 31/4985

(57) ABSTRACT

The present invention relates to solid oral pharmaceutical compositions for chronotropic administration of dipeptidyl peptidase IV inhibitors consisting of a monolithic matrix core comprising at least one low/medium viscosity hydroxypropyl methylcellulose, at least one medium/high viscosity hydroxypropyl methylcellulose, one or more methacrylic polymers or copolymers and/or cellulose acetate phthalate or shellac and an outer coating of said core consisting of a layer comprising ethylcellulose, a gastroresistant layer, or a layer comprising ethylcellulose coated in turn with gastroresistant polymers.

The present invention relates to solid oral pharmaceutical compositions for chronotropic administration of dipeptidyl peptidase IV inhibitors. The formulations according to the invention comprise the active ingredient in a core consisting of a monolithic matrix comprising at least one low/medium viscosity hydroxypropyl methylcellulose, at least one medium/high viscosity hydroxypropyl methylcellulose, one or more methacrylic polymers or copolymers and/or cellulose acetate phthalate or shellac, and an outer coating of said core consisting of a layer comprising ethylcellulose, or of a gastroresistant layer or a layer comprising ethylcellulose coated in turn with gastroresistant polymers.

### PRIOR ART

Dipeptidyl peptidase IV (DPP-4) inhibitors are a novel class of antihyperglycaemic medicaments. Sitagliptin, vildagliptin, saxagliptin and linagliptin belong to this class. Sitagliptin is a potent selective inhibitor of enzyme DPP-4, and does not inhibit the activity of the closely correlated enzymes DPP-8 or DPP-9 at therapeutic concentrations.

Recent studies have found that some classes of antidiabetics require targeted, time-controlled administration to optimise night-time blood glucose control (nocturnal hepatic gluconeogenesis).

During a short period of fasting, such as the interval between meals or overnight, the blood glucose is maintained within the normal range due to hepatic glycogenolysis and the release of fatty acids from the adipose tissue and ketone bodies from the liver. In addition to these general chronotropic mechanisms, dipeptidyl peptidase IV inhibitors, especially sitagliptin, prevent incretin hydrolysis by dipeptidyl peptidase IV (DPP-4), thereby increasing the plasma concentrations of the active forms of GLP-1 and GIP. By increasing the active incretin levels, said medicaments increase insulin release and reduce glucagon levels to a glucose-dependent extent. In patients suffering from type 2 diabetes, the receptors that regulate enzymatic activation of incretins are located partly in the duodenum and the proximal loops of the jejunum (GIP), and partly in the enteroendocrine L cells, located in the distal tract of the ileum and colon (GLP-1). The time- and site specific-administration in the gastrointestinal tract of said inhibitors therefore seems to have considerable therapeutic significance.

The rationale for optimising the quality, safety and efficacy of dipeptidyl peptidase IV inhibitors is to ensure that the active ingredient is carried to the specific site of action (colonic release), released gradually and consistently for a certain number of hours, and distributed homogeneously in the therapeutic system, with a reproducible release profile and a very low coefficient of standard deviation. The formulations of sitagliptin and other drugs of the same type currently on the market do not meet this requirement.

Various controlled-release formulations based on monolithic, multi-particulate or multi-unit matrix or reservoir systems have been described. The technologies used comprise gastroresistant retard systems; slow-release systems (simple matrices); solely pH-dependent release systems; solely pH-independent release systems; pulsatile-release systems (an immediate-release portion combined with a slow, gradual controlled-release portion with a simple matrix); extended-release systems (simple extended-release matrices); and reservoir systems involving the use of containment polymers, acting as semipermeable membranes.

The known formulations, which are described, for example, in WO201616770, WO201616772, US20110311594 and WO2014167437 and mainly characterised by single-component systems or matrices comprising two different types of hydroxypropyl methylcellulose not combined with other types of polymers, offer low precision of release of the active ingredient into the site and over time, and a high variability of release both in vitro and in vivo, as the release control effect is determined by a single type of excipient. In particular, the compositions described in WO201616770 and WO201616772, as indicated by the dissolution data reported on p. 46, do not guarantee 110% release of sitagliptin after 24 hours, implying a potential reduction in activity and efficacy. In addition, the standard deviations of the various sampling points are high, often over 3, with some values reaching and exceeding values of 4, 6, 10 or even 13. This may indicate a lack of homogeneity of the plasma concentration of the medicament due to the high variability of release by the therapeutic system.

### DESCRIPTION OF THE INVENTION

It has now been found that the activity of dipeptidyl peptidase IV inhibitors, especially sitagliptin, can be efficiently modulated by reducing their frequency of administration and controlling their release in particular sites of the gastrointestinal tract, using complex matrices consisting of a combination of polymers with different characteristics.

In particular, it has been found that by combining at least two types of hydroxypropyl methylcellulose having different viscosities with methacrylic polymers or copolymers and/or cellulose resins or esters or shellac, formulations that eliminate the limitations of the previously known formulations can be prepared.

The solid oral controlled-release pharmaceutical compositions according to the invention comprise a core and an outer coating of said core, wherein:
a) the core consists of:
   (i) a monolithic matrix containing a dipeptidyl peptidase IV inhibitor, at least one hydroxypropyl methylcellulose having a viscosity ranging between 3 and 5000 mPa·s 2% in H₂O at 20° C., at least one hydroxypropyl methylcellulose having a viscosity ranging between 13500 and 280000 mPa·s 2% in H₂O at 20° C., at least one or more methacrylic polymers/copolymers and/or shellac, cellulose acetate phthalate, hydroxypropyl methylcellulose acetate succinate;
   (ii) a monolithic matrix as defined above adjacent to an immediate-release layer comprising a dipeptidyl peptidase IV inhibitor;
b) the coating consists of a layer comprising hydroxypropyl methylcellulose and/or ethylcellulose, or of a gastroresistant layer or a layer comprising ethylcellulose which, in turn, is coated with gastroresistant polymers.

The core can consist of a monolithic matrix (i) or a bi-layer system consisting of a monolithic matrix (i) adjacent to an immediate-release layer comprising a dipeptidyl peptidase IV inhibitor.

The coating consists of a layer comprising hydroxypropyl methylcellulose and/or ethylcellulose or, in another embodiment of the invention, coating b) consists of a layer comprising ethylcellulose coated with gastroresistant polymers.

In yet another embodiment of the invention, the coating consists of a gastroresistant layer. Examples of dipeptidyl peptidase IV inhibitors comprise sitagliptin, vildagliptin, saxagliptin and linagliptin.

The acrylic/methacrylic polymers or copolymers of matrix (i) are preferably selected from copolymers of pH-independent methacrylic esters, pH-independent ammonium alkyl methacrylate copolymers; amino alkyl methacrylate copolymers soluble up to pH 5.0, methacrylic acid copolymers soluble at pH≥5.5, methacrylic acid copolymers soluble at pH 6.0-7.0; and pH-dependent methacrylic acid copolymers soluble at pH≥7.0.

According to one embodiment of the invention, the acrylic polymers or copolymers are combined with each other or a shellac; or the latter can replace said acrylic polymers/copolymers.

The gastroresistant coating can be the conventional type, and typically comprises methacrylic acid copolymers soluble at pH≥5.5. Examples of said copolymers are available on the market (Eudragit). Preferably the combination of polymethacrylate L100 with polymethacrylate S100 at the ratio of 1:10-10:1 (preferably 1:1); or L100/55 soluble at pH≥5.5; or shellac; or cellulose acetate phthalates/succinates is used.

In the compositions according to the invention, the hydroxypropyl methylcellulose having a viscosity ranging between 3 and 5000 mPa·s 2% in H₂O at 20° C. constitutes 1 to 20% of the weight of the core, the hydroxypropyl methylcellulose having a viscosity ranging between 13500 and 280000 mPa·s 2% in H₂O at 20° C. constitutes 1 to 20% of weight of the matrix, and the methacrylic polymer/copolymer constitutes 0.1 to 20% of the weight of the core.

Hydroxypropyl methylcellulose having a viscosity ranging between 3.0 and 5000 mPa·s 2% in H₂O at 20° C. is available on the market under the names of Methocel K3LV and K100 LV, K4M.

Hydroxypropyl methylcellulose having a viscosity ranging between 13500 and 280000 mPa·s 2% in H₂O at 20° C. is available on the market under the names of Methocel K15M, K100 M and K200M.

Hydroxypropyl methylcellulose can be present in the core-coating layer in percentages ranging from 1% to 20% of the weight of the core; preferably 5%.

Ethylcellulose is present in the core-coating layer in percentages ranging from 1% to 20% of the weight of the core; preferably 5%.

The matrix core can comprise conventional excipients such as diluents (microcrystalline cellulose, starches, sugars), binders (PVP, starches, cellulose, dextrins, maltodextrins, low-viscosity cellulose), glidants (colloidal silicas) flow agents (talc), lubricants (Mg stearate, fumaryl stearate, stearic acid, glyceryl behenate) disintegrating agents (croscarmellose, sodium starch glycolate, crosslinked polyvinylpyrrolidone, starches) and other functional excipients (waxes, polycarbophil, carbomer, glycerides).

The matrix is prepared by processes of partition and direct compression, dry granulation, compacting, wet granulation, melting and extrusion.

The resulting matrix/mini-matrix can then be coated with a gastroresistant film containing pH-dependent polymers that prevent release for at least 2 hours under acid pH conditions. The following can be used for this purpose: pH-dependent methacrylic acid copolymers soluble at pH≥5.5 (L100-55/L 30 D-55); pH-dependent methacrylic acid copolymers soluble at pH 6.0-7.0 (L100/L 12.5); pH-dependent methacrylic acid copolymers soluble at pH≥7.0 (S100/S 12.5/FS 30D); shellac; cellulose acetate phthalate; hydroxypropyl methylcellulose acetate succinate.

At a third stage, a core coating can be applied which is alternative and/or additional to and beneath the gastroresistant coating with pH-independent polymers (ethylcellulose or hydroxypropyl methylcellulose with different viscosities), which act as membranes delaying the passage of the ingredient loaded into the matrix/mini-matrix core following contact with biological fluids.

The matrix is coated with a quantity of polymer sufficient to guarantee that it remains intact in gastric and enteric juices for at least 2-4 hours before the release of the active ingredient from the core (lag time). To reduce the impact of the variability of gastric voiding times, the formulations can include a further gastroresistant coating (pH-dependent) outside the matrix core (pH-independent) and outside the cellulose film coating (pH-independent), to further delay contact between the biological fluids and the modified-release core (extended release).

In this way the system prevents early release during the stomach-jejunum transit time, initiating the modulated-release programme lasting up to 24 hours and ensuring homogenous distribution of the active ingredient in the duodenum, ileum and distal ileum and in the ascending, transverse and descending tracts of the large intestine.

The use of hydrophilic polymers with different rheological characteristics (viscosity/swelling properties) combined with pH-dependent and/or pH-independent polymers allows the release to be modulated for between 8 and 24 hours. If desired, a modified-, controlled-release core can be combined with an immediate-release layer (bi-layer and/or tri-layer matrix/mini-matrix); a system thus designed gives results of “therapeutic equivalence” or different levels of therapeutic efficacy.

The invention is described in greater detail in the examples below.

### EXAMPLE 1

1 Kg of sitagliptin is loaded into a granulator with 1.9 Kg of calcium phosphate, 450 g of microcrystalline cellulose, 500 g of hydroxypropyl methylcellulose (HPMC K4M), 400 g of hydroxypropyl methylcellulose (HPMC K100M) and 9 g of polymethacrylate L100-55. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a tablet weighing 433 mg. The resulting tablets are film-coated with a gastroresistant solution/suspension based on 169.4 g of polymethacrylate L100-55, 86.1 g of talc, 29 g of titanium dioxide and 15.5 g of triethyl citrate, to obtain a tablet with a mean weight of 463 mg.

When subjected to disintegration and dissolution tests at pH 1, the tablets remain intact for at least 2 hours, with release below 1%; when subjected to the dissolution test at pH≥6.4 they exhibit the following release profile: not more than 15% after 1 hour, at pH 7.2 not more than 30% after 1 hour, and not more than 50% after 2 hours; the value must be >80% after 6 hours; and 100% after 10 hours.

### EXAMPLE 2

1 Kg of sitagliptin is loaded into a granulator with 1.9 Kg of calcium phosphate, 450 g of microcrystalline cellulose, 500 g of hydroxypropyl methylcellulose (HPMC K4M), 400 g of hydroxypropyl methylcellulose (HPMC K100M), 4.5 g of polymethacrylate L100 and 4.5 g of polymethacrylate S100. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a tablet weighing 433 mg. The resulting tablets are film-coated with a gastroresistant solution/suspension based on 84.7 g of polymethacrylate L100, 84.7 g of polymethacrylate S100, 86.1 g of talc, 29 g of titanium dioxide and 15.5 g of triethyl citrate, to obtain a tablet with a mean weight of 463 mg.

When subjected to disintegration and dissolution tests at pH 1, the tablets remain intact for at least 2 hours, with release below 1%; when subjected to the dissolution test at pH≥6.4 they exhibit the following release profile: not more than 1% after 1 hour, at pH 7.2 not more than 30% after 1 hour, and not more than 50% after 2 hours; the value must be >80% after 6 hours; and 100% after 10 hours.

### EXAMPLE 3

1 Kg of sitagliptin is loaded into a granulator with 1.9 Kg of calcium phosphate, 450 g of microcrystalline cellulose, 500 g of hydroxypropyl methylcellulose (HPMC K4M), 400 g of hydroxypropyl methylcellulose (HPMC K100M) and 9 g of polymethacrylate L100-55. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a tablet weighing 433 mg. The resulting tablets are film-coated, firstly with a solution/suspension of 200 g of ethylcellulose, 28.05 g of talc and 6.45 g of triethyl citrate, and then with a gastroresistant solution/suspension based on 169.4 g of polymethacrylate L100-55, 43 g of talc, 14.5 g of titanium dioxide and 6.45 g of triethyl citrate, to obtain a tablet with a mean weight of 480 mg.

When subjected to disintegration and dissolution tests at pH 1, the tablets remain intact for at least 2 hours, with release below 1%; when subjected to the dissolution test at pH≥6.4 they exhibit the following release profile: not more than 1% after 5 hour, at pH 7.2 not more than 20% after 1 hour, and not more than 40% after 2 hours; the value must be >85% after 6 hours; and 100% after 18 hours.

### EXAMPLE 4

500 g of sitagliptin is loaded into a granulator with 900 g of calcium phosphate and 350 g of microcrystalline cellulose. 250 g of hydroxypropyl methylcellulose (HPMC K4M), 400 g of hydroxypropyl methylcellulose (HPMC K100 M) and 4.5 g of polymethacrylate L100-55 are added in sequence to the resulting mixture; the ingredients are mixed until a homogeneous dispersion of active ingredient in the matrices is obtained. 25.5 g of talc and 15 g of magnesium stearate are then added in sequence. The mixture is then homogenised for at least 15 minutes. This mixture will form part of the first, controlled-release layer of the tablet.

500 g of sitagliptin is loaded into a second granulator. 111.5 g of microcrystalline cellulose, 500 g of calcium phosphate, 250 g of crospovidone, 200 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 385 mg. The resulting tablets are film-coated with a gastroresistant solution/suspension of 169.4 g of polymethacrylate L100-55, 86.1 g of talc, 29 g of titanium dioxide and 15.5 g of triethyl citrate, to obtain a tablet with a mean weight of 415 mg.

When subjected to disintegration and dissolution tests at pH 1, the tablets remain intact for at least 2 hours, with release below 1%; when subjected to the dissolution test at pH≥6.4 they exhibit the following release profile: not more than 10% after 1 hour, at pH 7.2 not more than 50% after 1 hour, and not more than 60% after 2 hours; not more than 75% after 6 hours; less than 85% after 8 hours; less than 95% after 10 hours; and 100% after 18 hours.

### EXAMPLE 5

500 g of sitagliptin is loaded into a granulator with 900 g of calcium phosphate and 350 g of microcrystalline cellulose. 250 g of hydroxypropyl methylcellulose (HPMC K100 1v), 200 g of hydroxypropyl methylcellulose (HPMC K15 M), 2.25 g of polymethacrylate L100 and 2.25 g of polymethacrylate S100 are added in sequence to the resulting mixture. The ingredients are mixed until a homogeneous dispersion of active ingredient in the matrices is obtained. 22.5 g of talc and 13 g of magnesium stearate are then added in sequence. The mixture is then homogenised for at least 15 minutes. This mixture will form part of the first, controlled-release layer of the tablet.

500 g of sitagliptin is loaded into a second granulator. 111.5 g of microcrystalline cellulose, 500 g of calcium phosphate, 250 g of crospovidone, 200 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 385 mg. The resulting tablets are film-coated with a gastroresistant solution/suspension of 87.4 g of polymethacrylate L100, 87.4 g of polymethacrylate S100, 86.1 g of talc, 29 g of titanium dioxide and 15.5 g of triethyl citrate, to obtain a tablet with a mean weight of 415 mg.

When subjected to disintegration and dissolution tests at pH 1, the tablets remain intact for at least 2 hours, with release below 1%; when subjected to the dissolution test at pH≥6.4 they exhibit the following release profile: not more than 5% after 1 hour, at pH 7.2 not more than 50% after 1 hour, and not more than 60% after 2 hours; not more than 75% after 6 hours; less than 85% after 8 hours; less than 95% after 10 hours; and 100% after 18 hours.

### EXAMPLE 6

500 g of sitagliptin is loaded into a granulator with 900 g of calcium phosphate and 350 g of microcrystalline cellulose. 250 g of hydroxypropyl methylcellulose (HPMC K4M), 200 g of hydroxypropyl methylcellulose (HPMC K100 M) and 4.5 g of polymethacrylate L100-55 are added in sequence to the resulting mixture; the ingredients are mixed until a homogeneous dispersion of active ingredient in the matrices is obtained. 22.5 g of talc and 13 g of magnesium stearate are then added in sequence. The mixture is then homogenised for at least 15 minutes. This mixture will form part of the first, controlled-release layer of the tablet.

500 g of sitagliptin is loaded into a second granulator. 111.5 g of microcrystalline cellulose, 500 g of calcium phosphate, 250 g of crospovidone, 200 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 385 mg. The resulting tablets are film-coated, firstly with a solution/suspension of 200 g of ethylcellulose, 28.05 g of talc and 7.75 g of triethyl citrate, and then with a gastroresistant solution/suspension of 169.4 g of polymethacrylate L100-55, 28.05 g of talc, 14.5 g of titanium dioxide and 7.75 g of triethyl citrate, to obtain a tablet with a mean weight of 432 mg.

When subjected to disintegration and dissolution tests at pH 1, the tablets remain intact for at least 2 hours, with release below 1%; when subjected to the dissolution test at pH≥6.4 they exhibit the following release profile: not more than 3% after 1 hour, at pH 7.2 not more than 40% after 1 hour, and not more than 50% after 2 hours; not more than 70% after 6 hours; less than 80% after 8 hours; not more than 95% after 10 hours; and 100% after 18 hours.

### EXAMPLE 7

500 g of sitagliptin is loaded into a granulator with 1 Kg of calcium phosphate, 220 g of microcrystalline cellulose, 150 g of hydroxypropyl methylcellulose (HPMC K4M), 300 g of hydroxypropyl methylcellulose (HPMC K100M) and 4.5 g of polymethacrylate L100-55. The ingredients are mixed until a homogeneous dispersion of active ingredient in the matrices is obtained; 13 g of magnesium stearate and 22.5 g of talc are then added in sequence. The mixture is then homogenised for at least 15 minutes. This mixture is then compressed to obtain a tablet weighing 221.5 mg. The resulting tablets are film-coated with a gastroresistant solution/suspension based on 122.9 g of polymethacrylate L100-55, 57.4 g of talc, 19.3 g of titanium dioxide and 15.4 g of triethyl citrate, to obtain a tablet with a mean weight of 242.5 mg.

When subjected to disintegration and dissolution tests at pH 1, the tablets remain intact for at least 2 hours, with release below 1%; when subjected to the dissolution test at pH≥6.4 they exhibit the following release profile: not more than 15% after 1 hour, at pH 7.2 not more than 35% after 1 hour, and not more than 60% after 2 hours; the value must be >80% after 6 hours; and 100% after 10 hours.

### EXAMPLE 8

500 g of sitagliptin is loaded into a granulator with 1 Kg of calcium phosphate, 200 g of microcrystalline cellulose, 150 g of hydroxypropyl methylcellulose (HPMC K4M), 300 g of hydroxypropyl methylcellulose (HPMC K100M), 2.25 g of polymethacrylate L100 and 2.25 g of polymethacrylate S100. The ingredients are mixed until a homogeneous dispersion of active ingredient in the matrices is obtained; 13 g of magnesium stearate and 22.5 g of talc are then added in sequence. The mixture is then homogenised for at least 15 minutes. This mixture is then compressed to obtain a tablet weighing 221 mg. The resulting tablets are film-coated with a gastroresistant solution/suspension based on 61.45 g of polymethacrylate L100, 61.45 g of polymethacrylate S100, 57.4 g of talc, 19.3 g of titanium dioxide and 15.4 g of triethyl citrate, to obtain a tablet with a mean weight of 242.5 mg.

When subjected to disintegration and dissolution tests at pH 1, the tablets remain intact for at least 2 hours, with release below 1%; when subjected to the dissolution test at pH≥6.4 they exhibit the following release profile: not more than 1% after 1 hour, at pH 7.2 not more than 25% after 1 hour, not more than 40% after 2 hours; the value must be >80% after 6 hours; and 100% after 10 hours.

### EXAMPLE 9

500 g of sitagliptin is loaded into a granulator with 1 Kg of calcium phosphate, 220 g of microcrystalline cellulose, 150 g of hydroxypropyl methylcellulose (HPMC K4M), 300 g of hydroxypropyl methylcellulose (HPMC K100M) and 4.5 g of polymethacrylate L100-55. The ingredients are mixed until a homogeneous dispersion of active ingredient in the matrices is obtained; 13 g of magnesium stearate and 22.5 g of talc are then added in sequence. The mixture is then homogenised for at least 15 minutes. This mixture is then compressed to obtain a tablet weighing 221 mg. The resulting tablets are film-coated, firstly with a solution/suspension of 27 g of ethylcellulose, 28.7 g of talc and 7.7 g of triethyl citrate, and then with a gastroresistant solution/suspension based on 122.9 g of polymethacrylate L100-55, 28.7 g of talc, 19.3 g of titanium dioxide and 7.7 g of triethyl citrate, to obtain a tablet with a mean weight of 247.5 mg.

When subjected to disintegration and dissolution tests at pH 1, the tablets remain intact for at least 2 hours, with release below 1%; when subjected to the dissolution test at pH≥6.4 they exhibit the following release profile: not more than 1% after 1 hour, at pH 7.2 not more than 20% after 1 hour, and not more than 40% after 2 hours; the value must be >70% after 6 hours; >90% after 10 hours; and 100% after 18 hours.

### EXAMPLE 10

250 g of sitagliptin is loaded into a granulator with 800 g of calcium phosphate and 110 g of microcrystalline cellulose. 100 g of hydroxypropyl methylcellulose (HPMC K4M), 180 g of hydroxypropyl methylcellulose (HPMC K100 M) and 20 g of polymethacrylate L100-55 are added in sequence to the resulting mixture. The ingredients are mixed until a homogeneous dispersion of active ingredient in the matrices is obtained. 11 g of talc and 7 g of magnesium stearate are then added in sequence. The mixture is then homogenised for at least 15 minutes. This mixture will form part of the first, controlled-release layer of the tablet.

250 g of sitagliptin is loaded into a second granulator. 50 g of microcrystalline cellulose, 250 g of calcium phosphate, 150 g of crospovidone, 70 g of croscarmellose, 8 g of magnesium stearate and 22 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 226 mg. The resulting tablets are film-coated with a gastroresistant solution/suspension of 122.9 g of polymethacrylate L100-55, 57.4 g of talc, 19.3 g of titanium dioxide and 15.4 g of triethyl citrate, to obtain a tablet with a mean weight of 247.5 mg.

When subjected to disintegration and dissolution tests at pH 1, the tablets remain intact for at least 2 hours, with release below 1%; when subjected to the dissolution test at pH≥6.4 they exhibit the following release profile: not more than 50% after 1 hour, at pH 7.2 not more than 65% after 1 hour, and not more than 75% after 2 hours; not more than 80% after 6 hours; not more than 85% after 8 hours; and 100% after 10 hours.

### EXAMPLE 11

250 g of sitagliptin is loaded into a granulator with 800 g of calcium phosphate and 111 g of microcrystalline cellulose. 110 g of hydroxypropyl methylcellulose (HPMC K4M), 180 g of hydroxypropyl methylcellulose (HPMC K100 M), 10 g of polymethacrylate L100 and 10 g of polymethacrylate S100 are added in sequence to the resulting mixture. The ingredients are mixed until a homogeneous dispersion of active ingredient in the matrices is obtained. 11 g of talc and 7 g of magnesium stearate are then added in sequence. The mixture is then homogenised for at least 15 minutes. This mixture will form part of the first, controlled-release layer of the tablet.

250 g of sitagliptin is loaded into a second granulator. 50 g of microcrystalline cellulose, 250 g of calcium phosphate, 150 g of crospovidone, 70 g of croscarmellose, 8 g of magnesium stearate and 22 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 226 mg. The resulting tablets are film-coated with a gastroresistant solution/suspension of 61.45 g of polymethacrylate L100, 61.45 g of polymethacrylate S100, 57.4 g of talc, 19.3 g of titanium dioxide and 15.4 g of triethyl citrate, to obtain a tablet with a mean weight of 247.5 mg.

When subjected to disintegration and dissolution tests at pH 1, the tablets remain intact for at least 2 hours, with release below 1%; when subjected to the dissolution test at pH≥6.4 they exhibit the following release profile: not more than 1% after 1 hour, at pH 7.2 not more than 50% after 1 hour, and not more than 60% after 2 hours; not more than 70% after 6 hours; not more than 80% after 8 hours; not more than 90% after 10 hours; and 100% after 18 hours.

### EXAMPLE 12

250 g of sitagliptin is loaded into a granulator with 800 g of calcium phosphate and 110 g of microcrystalline cellulose. 110 g of hydroxypropyl methylcellulose (HPMC K100 1v), 180 g of hydroxypropyl methylcellulose (HPMC K15 M) and 20 g of polymethacrylate L100-55 are added in sequence to the resulting mixture. The ingredients are mixed until a homogeneous dispersion of active ingredient in the matrices is obtained. 11 g of talc and 7 g of magnesium stearate are then added in sequence. The mixture is then homogenised for at least 15 minutes. This mixture will form part of the first, controlled-release layer of the tablet.

250 g of sitagliptin is loaded into a second granulator. 50 g of microcrystalline cellulose, 250 g of calcium phosphate, 150 g of crospovidone, 70 g of croscarmellose, 8 g of magnesium stearate and 22 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 226 mg. The resulting tablets are film-coated, firstly with a solution/suspension of 120 g of ethylcellulose, 2.87 g of talc and 7.7 g of triethyl citrate, and then with a gastroresistant solution/suspension of 122.9 g of polymethacrylate L100-55, 19.3 g of titanium dioxide, 7.7 g of triethyl citrate and 2.87 g of talc, to obtain a tablet with a mean weight of 259.5 mg.

When subjected to disintegration and dissolution tests at pH 1, the tablets remain intact for at least 2 hours, with release below 1%; when subjected to the dissolution test at pH≥6.4 they exhibit the following release profile: not more than 5% after 1 hour, at pH 7.2 not more than 50% after 1 hour, and not more than 60% after 2 hours; not more than 70% after 6 hours; not more than 80% after 8 hours; not more than 90% after 10 hours; and 100% after 18 hours.

### EXAMPLE 13

250 g of sitagliptin is loaded into a granulator with 1.5 Kg of calcium phosphate, 225 g of microcrystalline cellulose, 150 g of hydroxypropyl methylcellulose (HPMC K4M), 300 g of hydroxypropyl methylcellulose (HPMC K100M) and 4.5 g of polymethacrylate L100-55. The ingredients are mixed until a homogeneous dispersion of active ingredient in the matrices is obtained; 13 g of magnesium stearate and 22.5 g of talc are then added in sequence. The mixture is then homogenised for at least 15 minutes. This mixture is then compressed to obtain a tablet weighing 221.5 mg. The resulting tablets are film-coated with a gastroresistant solution/suspension based on 122.9 g of polymethacrylate L100-55, 57.4 g of talc, 19.3 g of titanium dioxide and 15.4 g of triethyl citrate, to obtain a tablet with a mean weight of 243 mg.

When subjected to disintegration and dissolution tests at pH 1, the tablets remain intact for at least 2 hours, with release below 1%; when subjected to the dissolution test at pH≥6.4 they exhibit the following release profile: not more than 15% after 1 hour, at pH 7.2 not more than 35% after 1 hour, and not more than 60% after 2 hours; the value must be >80% after 6 hours; and 100% after 10 hours.

### EXAMPLE 14

250 g of sitagliptin is loaded into a granulator with 1.5 Kg of calcium phosphate, 225 g of microcrystalline cellulose, 150 g of hydroxypropyl methylcellulose (HPMC K4M), 300 g of hydroxypropyl methylcellulose (HPMC K100M), 2.25 g of polymethacrylate L100 and 2.25 g of polymethacrylate S100. The ingredients are mixed until a homogeneous dispersion of active ingredient in the matrices is obtained; 13 g of magnesium stearate and 22.5 g of talc are then added in sequence. The mixture is then homogenised for at least 15 minutes. This mixture is then compressed to obtain a tablet weighing 221.5 mg. The resulting tablets are film-coated with a gastroresistant solution/suspension based on 61.45 g of polymethacrylate L100, 61.45 g of polymethacrylate S100, 57.4 g of talc, 19.3 g of titanium dioxide and 15.4 g of triethyl citrate, to obtain a tablet with a mean weight of 243 mg.

When subjected to disintegration and dissolution tests at pH 1, the tablets remain intact for at least 2 hours, with release below 1%; when subjected to the dissolution test at pH≥6.4 they exhibit the following release profile: not more than 1% after 1 hour, at pH 7.2 not more than 30% after 1 hour, and not more than 60% after 2 hours; the value must be >80% after 6 hours; and 100% after 10 hours.

### EXAMPLE 15

250 g of sitagliptin is loaded into a granulator with 1.5 Kg of calcium phosphate, 225 g of microcrystalline cellulose, 150 g of hydroxypropyl methylcellulose (HPMC K4M), 300 g of hydroxypropyl methylcellulose (HPMC K100M) and 4.5 g of polymethacrylate L100-55. The ingredients are mixed until a homogeneous dispersion of active ingredient in the matrices is obtained; 13 g of magnesium stearate and 22.5 g of talc are then added in sequence. The mixture is then homogenised for at least 15 minutes. This mixture is then compressed to obtain a tablet weighing 221.5 mg. The resulting tablets are film-coated, firstly with a solution/suspension of 120 g of ethylcellulose, 2.87 g of talc and 7.7 g of triethyl citrate, and then with a gastroresistant solution/suspension of 122.9 g of polymethacrylate L100-55, 2.87 g of talc, 19.3 g of titanium dioxide and 7.75 g of triethyl citrate, to obtain a tablet with a mean weight of 255 mg.

When subjected to disintegration and dissolution tests at pH 1, the tablets remain intact for at least 2 hours, with release below 1%; when subjected to the dissolution test at pH≥6.4 they exhibit the following release profile: not more than 1% after 1 hour, at pH 7.2 not more than 10% after 1 hour, and not more than 40% after 2 hours; the value must be >70% after 6 hours; >90% after 10 hours; and 100% after 18 hours.

### EXAMPLE 16

125 g of sitagliptin is loaded into a granulator with 937.5 g of lactose and 170 g of microcrystalline cellulose. 100 g of hydroxypropyl methylcellulose (HPMC K4M), 170 g of hydroxypropyl methylcellulose (HPMC K100 M) and 20 g of polymethacrylate L100-55 are added in sequence to the resulting mixture. The ingredients are mixed until a homogeneous dispersion of active ingredient in the matrices is obtained. 11 g of talc and 7 g of magnesium stearate are then added in sequence. The mixture is then homogenised for at least 15 minutes. This mixture will form part of the first, controlled-release layer of the tablet.

125 g of sitagliptin is loaded into a second granulator. 50 g of microcrystalline cellulose, 312.5 g of calcium phosphate, 70 g of crospovidone, 150 g of croscarmellose, 8 g of magnesium stearate and 22 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 226 mg. The resulting tablets are film-coated with a gastroresistant solution/suspension of 122.9 g of polymethacrylate L100-55, 57.4 g of talc, 19.3 g of titanium dioxide and 15.4 g of triethyl citrate, to obtain a tablet with a mean weight of 247.5 mg.

When subjected to disintegration and dissolution tests at pH 1, the tablets remain intact for at least 2 hours, with release below 1%; when subjected to the dissolution test at pH≥6.4 they exhibit the following release profile: not more than 50% after 1 hour, at pH 7.2 not more than 60% after 1 hour, and not more than 70% after 2 hours; not more than 80% after 6 hours; not more than 85% after 8 hours; and 100% after 10 hours.

### EXAMPLE 17

125 g of sitagliptin is loaded into a granulator with 937.5 g of calcium phosphate and 170 g of microcrystalline cellulose. 100 g of hydroxypropyl methylcellulose (HPMC K4M), 170 g of hydroxypropyl methylcellulose (HPMC K100 M), 10 g of polymethacrylate L100 and 10 g of polymethacrylate S100 are added in sequence to the resulting mixture. The ingredients are mixed until a homogeneous dispersion of active ingredient in the matrices is obtained. 11 g of talc and 7 g of magnesium stearate are then added in sequence. The mixture is then homogenised for at least 15 minutes. This mixture will form part of the first, controlled-release layer of the tablet.

125 g of sitagliptin is loaded into a second granulator. 50 g of microcrystalline cellulose, 312.5 g of calcium phosphate, 70 g of crospovidone, 150 g of croscarmellose, 8 g of magnesium stearate and 22 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 226 mg. The resulting tablets are film-coated with a gastroresistant solution/suspension of 61.45 g of polymethacrylate L100, 61.45 g of polymethacrylate S100, 57.4 g of talc, 19.3 g of titanium dioxide and 15.4 g of triethyl citrate, to obtain a tablet with a mean weight of 247.5 mg. When subjected to disintegration and dissolution tests at pH 1, the tablets remain intact for at least 2 hours, with release below 1%; when subjected to the dissolution test at pH≥6.4 they exhibit the following release profile: not more than 5% after 1 hour, at pH 7.2 not more than 50% after 1 hour, and not more than 70% after 2 hours; not more than 80% after 6 hours; not more than 85% after 8 hours; and 100% after 10 hours.

### EXAMPLE 18

125 g of sitagliptin is loaded into a granulator with 937.5 g of calcium phosphate and 170 g of microcrystalline cellulose. 100 g of hydroxypropyl methylcellulose (HPMC K4M), 170 g of hydroxypropyl methylcellulose (HPMC K100 M) and 2 g of polymethacrylate L100-55 are added in sequence to the resulting mixture. The ingredients are mixed until a homogeneous dispersion of active ingredient in the matrices is obtained. 11 g of talc and 7 g of magnesium stearate are then added in sequence. The mixture is then homogenised for at least 15 minutes. This mixture will form part of the first, controlled-release layer of the tablet.

125 g of sitagliptin is loaded into a second granulator. 50 g of microcrystalline cellulose, 312.5 g of calcium phosphate, 70 g of crospovidone, 150 g of croscarmellose, 8 g of magnesium stearate and 22 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 226 mg. The resulting tablets are film-coated, firstly with an aqueous solution/suspension of 120 g of ethylcellulose, 7.7 g of triethyl citrate and 2.87 g of talc, and then with a gastroresistant solution/suspension of 122.9 g of polymethacrylate L100-55, 2.87 g of talc, 19.3 g of titanium dioxide and 7.7 g of triethyl citrate, to obtain a tablet with a mean weight of 259.5 mg.

When subjected to disintegration and dissolution tests at pH 1, the tablets remain intact for at least 2 hours, with release below 1%; when subjected to the dissolution test at pH≥6.4 they exhibit the following release profile: not more than 1% after 1 hour, at pH 7.2 not more than 50% after 1 hour, and not more than 70% after 2 hours; not more than 80% after 6 hours; not more than 85% after 8 hours; not more than 90% after 10 hours; and 100% after 18 hours.

### EXAMPLE 19

1 Kg of sitagliptin is loaded into a granulator with 450 g of calcium phosphate, 2 Kg of microcrystalline cellulose, 300 g of hydroxypropyl methylcellulose (HPMC K4M), 600 g of hydroxypropyl methylcellulose (HPMC K100M) and 9 g of polymethacrylate L100-55. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a mini-tablet weighing 44.3 mg. The resulting tablets are film-coated with a gastroresistant solution/suspension based on 169.4 g of polymethacrylate L100-55, 86.1 g of talc, 29 g of titanium dioxide and 15.5 g of triethyl citrate, to obtain a mini-tablet with a mean weight of 47.3 mg.

When subjected to disintegration and dissolution tests at pH 1, the mini-tablets remain intact for at least 2 hours, with release below 1%; when subjected to the dissolution test at pH≥6.4 they exhibit the following release profile: not more than 1% after 1 hour, at pH 7.2 not more than 30% after 1 hour, and not more than 60% after 2 hours; the value must be >80% after 6 hours; and 100% after 10 hours.

### EXAMPLE 20

1 Kg of sitagliptin is loaded into a granulator with 450 g of calcium phosphate, 2 Kg of microcrystalline cellulose, 300 g of hydroxypropyl methylcellulose (HPMC K4M), 600 g of hydroxypropyl methylcellulose (HPMC K100M), 4.5 g of polymethacrylate L100, and 4.5 g of polymethacrylate S100. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a mini-tablet weighing 44.3 mg. The resulting tablets are film-coated with a gastroresistant solution/suspension based on 84.7 g of polymethacrylate L100, 84.7 g of polymethacrylate S100, 86.1 g of talc, 29 g of titanium dioxide and 15.5 g of triethyl citrate, to obtain a mini-tablet with a mean weight of 47.3 mg.

When subjected to disintegration and dissolution tests at pH 1, the mini-tablets remain intact for at least 2 hours, with release below 1%; when subjected to the dissolution test at pH≥6.4 they exhibit the following release profile: not more than 1% after 1 hour, at pH 7.2 not more than 30% after 1 hour, and not more than 60% after 2 hours; the value must be >80% after 6 hours; and 100% after 10 hours.

### EXAMPLE 21

1 Kg of sitagliptin is loaded into a granulator with 450 g of calcium phosphate, 2 Kg of microcrystalline cellulose, 3000 g of hydroxypropyl methylcellulose (HPMC K4M), 600 g of hydroxypropyl methylcellulose (HPMC K100M) and 9 g of polymethacrylate L100-55. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a mini-tablet weighing 44.3 mg. The mini-tablets are film-coated, firstly with a solution/suspension of 27 g of ethylcellulose, 7.75 g of triethyl citrate and 43.05 g of talc, and then with a gastroresistant solution/suspension based on 169.4 g of polymethacrylate L100-55, 43.05 g of talc, 29 g of titanium dioxide and 7.75 g of triethyl citrate, to obtain a mini-tablet with a mean weight of 50 mg.

When subjected to disintegration and dissolution tests at pH 1, the mini-tablets remain intact for at least 2 hours, with release below 1%; when subjected to the dissolution test at pH≥6.4 they exhibit the following release profile: not more than 5% after 1 hour, at pH 7.2 not more than 50% after 1 hour, and not more than 60% after 2 hours; the value must be >80% after 6 hours; not more than 95% after 10 hours; and 100% after 18 hours.

### EXAMPLE 22

500 g of sitagliptin is loaded into a granulator with 225 g of calcium phosphate and 1 Kg of microcrystalline cellulose. 200 g of hydroxypropyl methylcellulose (HPMC K4M), 300 g of hydroxypropyl methylcellulose (HPMC K100 M) and 4.5 g of polymethacrylate L100-55 are added in sequence to the resulting mixture. The ingredients are mixed until a homogeneous dispersion of active ingredient in the matrices is obtained. 22.5 g of talc and 13 g of magnesium stearate are then added in sequence. The mixture is then homogenised for at least 15 minutes. This mixture will form part of the first layer of the controlled-release mini-tablet.

500 g of sitagliptin is loaded into a second granulator. 500 g of microcrystalline cellulose, 225 g of calcium phosphate, 250 g of crospovidone, 200 g of croscarmellose, 13 g of magnesium stearate and 27 g of talc are added and homogeneously mixed. The mixture is then homogenised for at least 15 minutes. This mixture will form part of the second layer of the immediate-release mini-tablet. The two separate mixtures are then compressed to obtain a double-layer mini-tablet weighing 39.80 mg. The resulting tablets are film-coated with a gastroresistant solution/suspension of 169.4 g of polymethacrylate L100-55, 86.1 g of talc, 29 g of titanium dioxide and 15.5 of triethyl citrate, to obtain a mini-tablet with a mean weight of 42.8 mg.

When subjected to disintegration and dissolution tests at pH 1, the tablets remain intact for at least 2 hours, with release below 1%; when subjected to the dissolution test at pH≥6.4 they exhibit the following release profile: not more than 50% after 1 hour, at pH 7.2 not more than 60% after 1 hour, and not more than 75% after 2 hours; not more than 80% after 6 hours; not more than 85% after 8 hours; and 100% after 10 hours.

### EXAMPLE 23

500 g of sitagliptin is loaded into a granulator with 225 g of calcium phosphate and 1 Kg of microcrystalline cellulose. 200 g of hydroxypropyl methylcellulose (HPMC K4M), 300 g of hydroxypropyl methylcellulose (HPMC K100 M), 2.25 g of polymethacrylate RL100, 2.25 g of polymethacrylate SL100 and 2.25 g of shellac are added in sequence to the resulting mixture. The ingredients are mixed until a homogeneous dispersion of active ingredient in the matrices is obtained. 22.5 g of talc and 13 g of magnesium stearate are then added in sequence. The mixture is then homogenised for at least 15 minutes. This mixture will form part of the first layer of the controlled-release mini-tablet.

500 g of sitagliptin is loaded into a second granulator. 500 g of microcrystalline cellulose, 225 g of calcium phosphate, 250 g of crospovidone, 200 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer mini-tablet weighing 39.82 mg. The resulting tablets are film-coated with a gastroresistant solution/suspension of 169.4 g of shellac, 83.85 g of talc, 29 g of titanium dioxide, 15.5 g of triethyl citrate and 20 g of hydroxypropyl methylcellulose E5 Premium to obtain a mini-tablet with a mean weight of 43 mg.

When subjected to disintegration and dissolution tests at pH 1, the tablets remain intact for at least 2 hours, with release below 1%; when subjected to the dissolution test at pH≥6.4 they exhibit the following release profile: not more than 10% after 1 hour, at pH 7.2 not more than 60% after 1 hour, and not more than 70% after 2 hours; not more than 80% after 6 hours; not more than 85% after 8 hours; and 100% after 10 hours.

### EXAMPLE 24

500 g of sitagliptin is loaded into a granulator with 225 g of calcium phosphate and 1 Kg of microcrystalline cellulose. 200 g of hydroxypropyl methylcellulose (HPMC K100 1v), 300 g of hydroxypropyl methylcellulose (HPMC K15 M) and 45 g of polymethacrylate L100-55 are added in sequence to the resulting mixture. The ingredients are mixed until a homogeneous dispersion of active ingredient in the matrices is obtained. 22.5 g of talc and 13 g of magnesium stearate are then added in sequence. The mixture is then homogenised for at least 15 minutes. This mixture will form part of the first layer of the controlled-release mini-tablet.

500 g of sitagliptin is loaded into a second granulator. 500 g of microcrystalline cellulose, 225 g of calcium phosphate, 250 g of crospovidone, 200 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer mini-tablet weighing 39.80 mg. The resulting tablets are film-coated with an aqueous solution/suspension of 270 g of ethylcellulose, 7.75 g of triethyl citrate and 43.05 of talc, and then with a gastroresistant solution/suspension of 169.4 g of Polymethacrylate L100-55, 43.05 g of talc, 29 g of titanium dioxide and 7.75 g of triethyl citrate until a mini-tablet with a mean weight of 45.50 mg is obtained.

When subjected to disintegration and dissolution tests at pH 1, the tablets remain intact for at least 2 hours, with release below 1%; when subjected to the dissolution test at pH≥6.4 they exhibit the following release profile: not more than 5% after 1 hour, at pH 7.2 not more than 50% after 1 hour, and not more than 70% after 2 hours; not more than 80% after 6 hours; not more than 85% after 8 hours; not more than 95% after 10 hours; and 100% after 18 hours.

### EXAMPLE 25

500 g of sitagliptin is loaded into a granulator with 950 g of calcium phosphate, 2.4 Kg of microcrystalline cellulose, 300 g of hydroxypropyl methylcellulose (HPMC K4M), 200 g of hydroxypropyl methylcellulose (HPMC K100M) and 9 g of polymethacrylate L100-55. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a mini-tablet weighing 44.3 mg. The resulting tablets are film-coated with a gastroresistant solution/suspension based on 169.4 g of polymethacrylate L100-55, 86.1 g of talc, 29 g of titanium dioxide and 15.5 g of triethyl citrate, to obtain a mini-tablet with a mean weight of 47.3 mg.

When subjected to disintegration and dissolution tests at pH 1, the mini-tablets remain intact for at least 2 hours, with release below 1%; when subjected to the dissolution test at pH≥6.4 they exhibit the following release profile: not more than 20% after 1 hour, at pH 7.2 not more than 30% after 1 hour, and not more than 60% after 2 hours; the value must be >80% after 6 hours; and 100% after 10 hours.

### EXAMPLE 26

500 g of sitagliptin is loaded into a granulator with 950 g of calcium phosphate, 2.4 Kg of microcrystalline cellulose, 300 g of hydroxypropyl methylcellulose (HPMC K4M), 200 g of hydroxypropyl methylcellulose (HPMC K100M), 4.5 g of polymethacrylate L100 and 4.5 g of polymethacrylate S100. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a mini-tablet weighing 44.3 mg. The resulting tablets are film-coated with a gastroresistant solution/suspension based on 84.7 g of polymethacrylate L100, 84.7 g of polymethacrylate S100, 86.1 g of talc, 29 g of titanium dioxide and 15.5 g of triethyl citrate, to obtain a mini-tablet with a mean weight of 47.3 mg.

When subjected to disintegration and dissolution tests at pH 1, the mini-tablets remain intact for at least 2 hours, with release below 1%; when subjected to the dissolution test at pH≥6.4 they exhibit the following release profile: not more than 1% after 1 hour, at pH 7.2 not more than 35% after 1 hour, and not more than 65% after 2 hours; the value must be >80% after 6 hours; and 100% after 10 hours.

### EXAMPLE 27

500 Kg of sitagliptin is loaded into a granulator with 950 g of calcium phosphate, 2.4 Kg of microcrystalline cellulose, 300 g of hydroxypropyl methylcellulose (HPMC K4M), 200 g of hydroxypropyl methylcellulose (HPMC K100M) and 9 g of polymethacrylate L100-55. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a mini-tablet weighing 44.3 mg. The resulting tablets are film-coated, firstly with a solution/suspension of 27 g of ethylcellulose, 7.75 g of triethyl citrate and 43.05 g of talc, and then with a gastroresistant solution/suspension based on 169.4 g of polymethacrylate L100-55, 43.05 g of talc, 29 g of titanium dioxide and 7.75 g of triethyl citrate, to obtain a mini-tablet with a mean weight of 50 mg.

When subjected to disintegration and dissolution tests at pH 1, the mini-tablets remain intact for at least 2 hours, with release below 1%; when subjected to the dissolution test at pH≥6.4 they exhibit the following release profile: not more than 5% after 1 hour, at pH 7.2 not more than 60% after 1 hour, and not more than 70% after 2 hours; the value must be >80% after 6 hours; not more than 95% after 10 hours; and 100% after 18 hours.

### EXAMPLE 28

250 g of sitagliptin is loaded into a granulator with 475 g of lactose and 1.8 Kg of microcrystalline cellulose. 100 g of hydroxypropyl methylcellulose (HPMC K4M), 200 g of hydroxypropyl methylcellulose (HPMC K100 M) and 45 g of polymethacrylate L100-55 are added in sequence to the resulting mixture. The ingredients are mixed until a homogeneous dispersion of active ingredient in the matrices is obtained. 22.5 g of talc and 13 g of magnesium stearate are then added in sequence. The mixture is then homogenised for at least 15 minutes. This mixture will form part of the first layer of the controlled-release mini-tablet.

250 g of sitagliptin is loaded into a second granulator. 600 g of microcrystalline cellulose, 475 g of calcium phosphate, 250 g of crospovidone, 200 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer mini-tablet weighing 46.8 mg. The resulting tablets are film-coated with a gastroresistant solution/suspension of 169.4 g of polymethacrylate L100-55, 86.1 g of talc, 29 g of titanium dioxide and 15.5 g of triethyl citrate, to obtain a mini-tablet with a mean weight of 49.8 mg.

When subjected to disintegration and dissolution tests at pH 1, the tablets remain intact for at least 2 hours, with release below 1%; when subjected to the dissolution test at pH≥6.4 they exhibit the following release profile: not more than 50% after 1 hour, at pH 7.2 not more than 65% after 1 hour, and not more than 70% after 2 hours; not more than 80% after 6 hours; not more than 85% after 8 hours; and 100% after 10 hours.

### EXAMPLE 29

250 g of sitagliptin is loaded into a granulator with 475 g of calcium phosphate and 1.8 Kg of microcrystalline cellulose. 100 g of hydroxypropyl methylcellulose (HPMC K4M), 200 g of hydroxypropyl methylcellulose (HPMC K100 M), 2.25 g of polymethacrylate L100 and 2.25 g of polymethacrylate S100 are added in sequence to the resulting mixture. The ingredients are mixed until a homogeneous dispersion of active ingredient in the matrices is obtained. 22.5 g of talc and 13 g of magnesium stearate are then added in sequence. The mixture is then homogenised for at least 15 minutes. This mixture will form part of the first layer of the controlled-release mini-tablet.

250 g of sitagliptin is loaded into a second granulator. 60 g of microcrystalline cellulose, 475 g of calcium phosphate, 250 g of crospovidone, 200 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer mini-tablet weighing 49.8 mg. The resulting tablets are film-coated with a gastroresistant solution/suspension of 84.7 g of polymethacrylate L100, 84.7 g of polymethacrylate S100, 83.85 g of talc, 29 g of titanium dioxide and 15.5 g of triethyl citrate, to obtain a mini-tablet with a mean weight of 49.8 mg.

When subjected to disintegration and dissolution tests at pH 1, the tablets remain intact for at least 2 hours, with release below 1%; when subjected to the dissolution test at pH≥6.4 they exhibit the following release profile: not more than 20% after 1 hour, at pH 7.2 not more than 50% after 1 hour, and not more than 70% after 2 hours; not more than 80% after 6 hours; not more than 85% after 8 hours; and 100% after 10 hours.

### EXAMPLE 30

250 g of sitagliptin is loaded into a granulator with 475 g of lactose and 1.8 Kg of microcrystalline cellulose. 100 g of hydroxypropyl methylcellulose (HPMC K4 M), 200 g of hydroxypropyl methylcellulose (HPMC K100 M) and 45 g of polymethacrylate L100-55 are added in sequence to the resulting mixture. The ingredients are mixed until a homogeneous dispersion of active ingredient in the matrices is obtained. 22.5 g of talc and 13 g of magnesium stearate are then added in sequence. The mixture is then homogenised for at least 15 minutes. This mixture will form part of the first layer of the controlled-release mini-tablet.

250 g of sitagliptin is loaded into a second granulator. 60 g of microcrystalline cellulose, 475 g of calcium phosphate, 250 g of crospovidone, 200 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer mini-tablet weighing 46.8 mg. The resulting tablets are film-coated with an aqueous solution/suspension of 270 g of ethylcellulose, 7.75 g of triethyl citrate and 43.05 of talc, and then with a gastroresistant solution/suspension of 169.4 g of polymethacrylate L100-55, 43.05 g of talc, 29 g of titanium dioxide and 7.75 g of triethyl citrate until a mini-tablet with a mean weight of 52.5 mg is obtained. When subjected to disintegration and dissolution tests at pH 1, the tablets remain intact for at least 2 hours, with release below 1%; when subjected to the dissolution test at pH≥6.4 they exhibit the following release profile: not more than 5% after 1 hour, at pH 7.2 not more than 40% after 1 hour, and not more than 70% after 2 hours; not more than 80% after 6 hours; not more than 85% after 8 hours; not more than 95% after 10 hours; and 100% after 18 hours.

### EXAMPLE 31

1 Kg of sitagliptin is loaded into a granulator with 1.9 Kg of calcium phosphate, 450 g of microcrystalline cellulose, 400 g of hydroxypropyl methylcellulose (HPMC K4M), 500 g of hydroxypropyl methylcellulose (HPMC K100M), 4.5 g of polymethacrylate RS100 and 4.5 g of polymethacrylate RL100. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a tablet weighing 433 mg. The resulting tablets are then film-coated with a solution/suspension based on 160 g of HPMC E5 Premium, 80 g of talc, 10 g of titanium dioxide and 20 g of triethyl citrate, to obtain a tablet with a mean weight of 460 mg.

When subjected to a dissolution test in aqueous solution, the tablets exhibit the following release profile: not more than 15% after 1 hour, not more than 30% after 2 hours; not more than 50% after 4 hours; not more than 60% after 8 hours; not more than 70% after 12 hours; not more than 80% after 16 hours; not more than 95% after 20 hours; ≥98% after 24 hours.

### EXAMPLE 32

1 Kg of sitagliptin is loaded into a granulator with 1.9 Kg of calcium phosphate, 450 g of microcrystalline cellulose, 400 g of hydroxypropyl methylcellulose (HPMC K4M), 500 g of hydroxypropyl methylcellulose (HPMC K100M), 4.5 g of polymethacrylate L100 and 4.5 g of polymethacrylate S100. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a tablet weighing 433 mg. The resulting tablets are then film-coated with a solution/suspension based on 160 g of HPMC E5 Premium, 80 g of talc, 10 g of titanium dioxide and 20 g of triethyl citrate, to obtain a tablet with a mean weight of 460 mg.

When subjected to a dissolution test in aqueous solution, the tablets exhibit the following release profile: not more than 15% after 1 hour, not more than 30% after 2 hours; not more than 50% after 4 hours; not more than 60% after 8 hours; not more than 75% after 12 hours; not more than 85% after 16 hours; not more than 95% after 20 hours; ≥98% after 24 hours.

### EXAMPLE 33

1 Kg of sitagliptin is loaded into a granulator with 1.9 Kg of calcium phosphate, 450 g of microcrystalline cellulose, 400 g of hydroxypropyl methylcellulose (HPMC K4M), 500 g of hydroxypropyl methylcellulose (HPMC K100M), 4.5 g of polymethacrylate L100 and 4.5 g of polymethacrylate S100. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a tablet weighing 433 mg. The resulting tablets are then film-coated with a solution/suspension based on 190 g of ethylcellulose, 80 g of talc, 10 g of titanium dioxide and 20 g of triethyl citrate, to obtain a tablet with a mean weight of 460 mg.

When subjected to a dissolution test in aqueous solution, the tablets exhibit the following release profile: not more than 15% after 1 hour, not more than 30% after 2 hours; not more than 50% after 4 hours; not more than 60% after 8 hours; not more than 75% after 12 hours; not more than 85% after 16 hours; not more than 95% after 20 hours; ≥98% after 24 hours.

### EXAMPLE 34

500 g of sitagliptin is loaded into a granulator with 900 g of calcium phosphate and 350 g of microcrystalline cellulose. 150 g of hydroxypropyl methylcellulose (HPMC K4M), 300 g of hydroxypropyl methylcellulose (HPMC K100 M), 2.25 g of polymethacrylate RL100 and 2.25 g of polymethacrylate RS100 are added in sequence to the resulting mixture, and the ingredients are mixed until a homogeneous dispersion of active ingredient in the matrices is obtained. 25.5 g of talc and 15 g of magnesium stearate are then added in sequence. The mixture is then homogenised for at least 15 minutes. This mixture will form part of the first, controlled-release layer of the tablet.

500 g of sitagliptin is loaded into a second granulator. 111.5 g of microcrystalline cellulose, 500 g of calcium phosphate, 250 g of crospovidone, 200 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 385 mg. The resulting tablets are then film-coated with a solution/suspension based on 220 g of HPMC E5 Premium, 50 g of talc, 10 g of titanium dioxide and 20 g of triethyl citrate, to obtain a tablet with a mean weight of 415 mg.

When subjected to a dissolution test in aqueous solution, the tablets exhibit the following release profile: not more than 55% after 1 hour, not more than 60% after 2 hours; not more than 65% after 4 hours; not more than 70% after 8 hours; not more than 85% after 12 hours; not more than 95% after 16 hours; not more than 98% after 20 hours; ≥99% after 24 hours.

### EXAMPLE 35

500 g of sitagliptin is loaded into a granulator with 900 g of calcium phosphate and 350 g of microcrystalline cellulose. 200 g of hydroxypropyl methylcellulose (HPMC K100 lv, 250 g of hydroxypropyl methylcellulose (HPMC K15 M), 2.25 g of polymethacrylate L100 and 2.25 g of polymethacrylate S100 are added in sequence to the resulting mixture, and the ingredients are mixed until a homogeneous dispersion of active ingredient in the matrices is obtained. 25.5 g of talc and 15 g of magnesium stearate are then added in sequence. The mixture is then homogenised for at least 15 minutes. This mixture will form part of the first layer of the 224.5 mg controlled-release tablet.

500 g of sitagliptin is loaded into a second granulator. 111.5 g of microcrystalline cellulose, 500 g of calcium phosphate, 250 g of crospovidone, 200 g of croscarmellose, 13 g of magnesium stearate and 27 g of talc are added and homogeneously mixed. The mixture is then homogenised for at least 15 minutes. This mixture will form part of the second layer of the 160.5 mg immediate-release tablet. The two separate mixtures are then compressed to obtain a double-layer tablet weighing 385 mg. The resulting tablets are then film-coated with a solution/suspension based on 220 g of HPMC E5 Premium, 50 g of talc, 10 g of titanium dioxide and 20 g of triethyl citrate, to obtain a tablet with a mean weight of 415 mg.

When subjected to a dissolution test in aqueous solution, the tablets exhibit the following release profile: not more than 55% after 1 hour, not more than 60% after 2 hours; not more than 65% after 4 hours; not more than 75% after 8 hours; not more than 85% after 12 hours; not more than 95% after 16 hours; not more than 98% after 20 hours; ≥99% after 24 hours.

### EXAMPLE 36

500 g of sitagliptin is loaded into a granulator with 900 g of calcium phosphate and 350 g of microcrystalline cellulose. 200 g of hydroxypropyl methylcellulose (HPMC K4M), 2500 g of hydroxypropyl methylcellulose (HPMC K100 M), 2.25 g of polymethacrylate RL100 and 2.25 g of polymethacrylate RS100 are added in sequence to the resulting mixture, and the ingredients are mixed until a homogeneous dispersion of active ingredient in the matrices is obtained. 25.5 g of talc and 15 g of magnesium stearate are then added in sequence. The mixture is then homogenised for at least 15 minutes. This mixture will form part of the first layer of the 224.5 mg controlled-release tablet.

500 g of sitagliptin is loaded into a second granulator. 111.5 g of microcrystalline cellulose, 500 g of calcium phosphate, 250 g of crospovidone, 200 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 385 mg. The resulting tablets are then film-coated with a solution/suspension based on 190 g of ethylcellulose, 70 g of talc, 15 g of titanium dioxide and 25 g of triethyl citrate, to obtain a tablet with a mean weight of 415 mg.

When subjected to a dissolution test in aqueous solution, the tablets exhibit the following release profile: not more than 55% after 1 hour, not more than 60% after 2 hours; not more than 65% after 4 hours; not more than 70% after 8 hours; not more than 85% after 12 hours; not more than 95% after 16 hours; not more than 98% after 20 hours; ≥99% after 24 hours.

### EXAMPLE 37

50 g of saxagliptin is loaded into a granulator with 1.9 Kg of calcium phosphate, 450 g of microcrystalline cellulose, 400 g of hydroxypropyl methylcellulose (HPMC K4M), 500 g of hydroxypropyl methylcellulose (HPMC K100M), 4.5 g of polymethacrylate RS100 and 4.5 g of polymethacrylate RL100. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a tablet weighing 338 mg. The resulting tablets are then film-coated with a solution/suspension based on 160 g of HPMC E5 Premium, 80 g of talc, 10 g of titanium dioxide and 20 g of triethyl citrate, to obtain a tablet with a mean weight of 365 mg.

When subjected to a dissolution test in aqueous solution, the tablets exhibit the following release profile: not more than 20% after 1 hour, not more than 30% after 2 hours; not more than 50% after 4 hours; not more than 60% after 8 hours; not more than 70% after 12 hours; not more than 85% after 16 hours; not more than 95% after 20 hours; ≥99% after 24 hours.

### EXAMPLE 38

50 g of linagliptin is loaded into a granulator with 1.9 Kg of calcium phosphate, 450 g of microcrystalline cellulose, 400 g of hydroxypropyl methylcellulose (HPMC K4M), 500 g of hydroxypropyl methylcellulose (HPMC K100M), 4.5 g of polymethacrylate RS100 and 4.5 g of polymethacrylate RL100. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a tablet weighing 338 mg. The resulting tablets are then film-coated with a solution/suspension based on 160 g of HPMC E5 Premium, 80 g of talc, 10 g of titanium dioxide and 20 g of triethyl citrate, to obtain a tablet with a mean weight of 365 mg.

When subjected to a dissolution test in aqueous solution, the tablets exhibit the following release profile: not more than 15% after 1 hour, not more than 25% after 2 hours; not more than 45% after 4 hours; not more than 60% after 8 hours; not more than 70% after 12 hours; not more than 85% after 16 hours; not more than 95% after 20 hours; ≥99% after 24 hours.

### EXAMPLE 39

500 g of vildagliptin is loaded into a granulator with 1.9 Kg of calcium phosphate, 450 g of microcrystalline cellulose, 400 g of hydroxypropyl methylcellulose (HPMC K4M), 500 g of hydroxypropyl methylcellulose (HPMC K100M), 4.5 g of polymethacrylate RS100 and 4.5 g of polymethacrylate RL100. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a tablet weighing 383 mg. The resulting tablets are then film-coated with a solution/suspension based on 160 g of HPMC E5 Premium, 80 g of talc, 10 g of titanium dioxide and 20 g of triethyl citrate, to obtain a tablet with a mean weight of 3410 mg.

When subjected to a dissolution test in aqueous solution, the tablets exhibit the following release profile: not more than 15% after 1 hour, not more than 25% after 2 hours; not more than 45% after 4 hours; not more than 60% after 8 hours; not more than 70% after 12 hours; not more than 88% after 16 hours; not more than 95% after 20 hours; ≥99% after 24 hours.

The following tables summarise the qualitative and quantitative compositions of examples 1-30.

TABLE 1
Sitagliptin 100 mg tablet

| | F1 | F2 | F3 | F4 | F5 | F6 |
|---|---|---|---|---|---|---|
| Ingredients of MR core | | | | | | |
| Sitagliptin | 100 | 100 | 100 | 50 | 50 | 50 |
| Calcium Phosphate | 190 | 190 | 190 | 90 | 90 | 90 |
| Microcrystalline cellulose | 45 | 45 | 45 | 35 | 35 | 35 |
| HPMC 100 lv | | | | | 25 | |
| HPMC K4M | 50 | 50 | 50 | 25 | | 25 |
| HPMC 15M | | | | | 20 | |
| HPMC K100M | 40 | 40 | 40 | 20 | | 20 |
| Eudragit L100/55 | 0.9 | | 0.9 | 0.45 | | 0.45 |
| Eudragit L100 | | 0.45 | | | 0.225 | |
| Eudragit S100 | | 0.45 | | | 0.225 | |
| Talc | 4.5 | 4.5 | 4.5 | 2.55 | 2.55 | 2.55 |
| Mg stearate | 2.6 | 2.6 | 2.6 | 1.5 | 1.5 | 1.5 |
| | 433 | 433 | 433 | 224.5 | 224.5 | 224.5 |
| Ingredients of IR core | | | | | | |
| Sitagliptin phosphate anhydrous | | | | 50 | 50 | 50 |
| Calcium phosphate | | | | 50 | 50 | 50 |
| Microcrystalline cellulose | | | | 11.5 | 11.5 | 11.5 |
| Crosslinked PVP | | | | 25 | 25 | 25 |
| AcDisol | | | | 20 | 20 | 20 |
| Talc | | | | 2.7 | 2.7 | 2.7 |
| Mg stearate | | | | 1.3 | 1.3 | 1.3 |
| Total | | | | 160.5 | 160.5 | 160.5 |
| Film-coating ingredients | | | | | | |
| Talc | 8.61 | 8.61 | 5.61 | 8.61 | 8.61 | 5.61 |
| Eudragit L100/55 | 16.94 | | 16.94 | 16.94 | | 16.94 |
| Eudragit L100 | | 8.47 | | | 8.47 | |
| Eudragit S100 | | 8.47 | | | 8.47 | |
| Ethylcellulose | | | 20 | | | 20 |
| Titanium dioxide | 2.90 | 2.90 | 2.90 | 2.90 | 2.90 | 2.90 |
| Triethyl citrate | 1.55 | 1.55 | 1.55 | 1.55 | 1.55 | 1.55 |
| Total | 30 | 30 | 47 | 30 | 30 | 47 |
| Grand total | 463 | 463 | 480 | 415 | 415 | 432 |

Sitagliptin phosphate anhydrous 27.914 (=25 mg);
sitagliptin phosphate anhydrous 55.828 (=50 mg);
Sitagliptin phosphate anhydrous 111.656 (=100 mg)

TABLE 2
Sitagliptin 50 mg tablet

| | F7 | F8 | F9 | F10 | F11 | F12 |
|---|---|---|---|---|---|---|
| Ingredients of MR core | | | | | | |
| Sitagliptin | 50 | 50 | 50 | 25 | 25 | 25 |
| Calcium phosphate | 100 | 100 | 100 | 80 | 80 | 80 |
| Microcrystalline cellulose | 22 | 22 | 22 | 11 | 11 | 11 |
| HPMC 100 lv | | | | | | 10 |
| HPMC K4M | 15 | 15 | 15 | 10 | 10 | |
| HPMC 15M | | | | | | 18 |
| HPMC K100M | 30 | 30 | 30 | 18 | 18 | |
| Eudragit L100/55 | 0.45 | | 0.45 | 0.2 | | 0.2 |
| Eudragit L100 | | 0.225 | | | 0.1 | |
| Eudragit S100 | | 0.225 | | | 0.1 | |
| Talc | 2.25 | 2.25 | 2.25 | 1.1 | 1.1 | 1.1 |
| Mg stearate | 1.3 | 1.3 | 1.3 | 0.7 | 0.7 | 0.7 |
| | 221 | 221 | 221 | 146 | 146 | 146 |
| Ingredients of IR core | | | | | | |
| Sitagliptin | | | | 25 | 25 | 25 |
| Calcium phosphate | | | | 25 | 25 | 25 |
| Microcrystalline cellulose | | | | 5 | 5 | 5 |
| Crosslinked PVP | | | | 15 | 15 | 15 |
| AcDisol | | | | 7 | 7 | 7 |
| Talc | | | | 2.2 | 2.2 | 2.2 |
| Mg stearate | | | | 0.8 | 0.8 | 0.8 |
| Total | | | | 80 | 80 | 80 |
| Film-coating ingredients | | | | | | |
| Talc | 5.74 | 5.74 | 5.74 | 5.74 | 5.74 | 5.74 |
| Eudragit L100/55 | 12.29 | | 12.29 | 12.29 | | 12.29 |
| Eudragit L100 | | 6.145 | | | 6.145 | |
| Eudragit S100 | | 6.145 | | | 6.145 | |
| Ethylcellulose | | | 12 | | | 12 |
| Titanium dioxide | 1.93 | 1.93 | 1.93 | 1.93 | 1.93 | 1.93 |
| Triethyl citrate | 1.54 | 1.54 | 1.54 | 1.54 | 1.54 | 1.54 |
| Total | 21.5 | 21.5 | 33.5 | 21.5 | 21.5 | 33.5 |
| Grand total | 242.5 | 242.5 | 254.5 | 247.5 | 247.5 | 259.5 |

TABLE 3
Sitagliptin 25 mg tablet

| | F13 | F14 | F15 | F16 | F17 | F18 |
|---|---|---|---|---|---|---|
| Ingredients of MR core | | | | | | |
| Sitagliptin | 25 | 25 | 25 | 12.5 | 12.5 | 12.5 |
| Calcium phosphate | 150 | 150 | 150 | 93.75 | 93.75 | 93.75 |
| Microcrystalline cellulose | 22.5 | 22.5 | 22.5 | 17 | 17 | 17 |
| HPMC K4M | 15 | 15 | 15 | 10 | 10 | 10 |
| HPMC K100M | 30 | 30 | 30 | 17 | 17 | 17 |
| Eudragit L100/55 | 0.45 | | 0.45 | 0.2 | | 0.2 |
| Eudragit L100 | | 0.225 | | | 0.1 | |
| Eudragit S100 | | 0.225 | | | 0.1 | |
| Talc | 2.25 | 2.25 | 2.25 | 1.1 | 1.1 | 1.1 |
| Mg stearate | 1.3 | 1.3 | 1.3 | 0.7 | 0.7 | 0.7 |
| | 221.5 | 221.5 | 221.5 | 152.25 | 152.25 | 152.25 |
| Ingredients of IR core | | | | | | |
| Sitagliptin | | | | 12.5 | 12.5 | 12.5 |
| Calcium phosphate | | | | 31.25 | 31.25 | 31.25 |
| Microcrystalline cellulose | | | | 5 | 5 | 5 |
| PVP | | | | 7 | 7 | 7 |
| AcDisol | | | | 15 | 15 | 15 |
| Talc | | | | 2.2 | 2.2 | 2.2 |
| Mg stearate | | | | 0.8 | 0.8 | 0.8 |
| Total | | | | 73.75 | 73.75 | 73.75 |
| Film-coating ingredients | | | | | | |
| Talc | 5.74 | 5.74 | 5.74 | 5.74 | 5.74 | 5.74 |
| Eudragit L100/55 | 12.29 | | 12.29 | 12.29 | | 12.29 |
| Eudragit L100 | | 6.145 | | | 6.145 | |
| Eudragit S100 | | 6.145 | | | 6.145 | |
| Ethylcellulose | | | 12 | | | 12 |
| Titanium dioxide | 1.93 | 1.93 | 1.93 | 1.93 | 1.93 | 1.93 |
| Triethyl citrate | 1.54 | 1.54 | 1.54 | 1.54 | 1.54 | 1.54 |
| Total | 21.5 | 21.5 | 33.5 | 21.5 | 21.5 | 33.5 |
| Grand total | 243 | 243 | 255 | 247.5 | 247.5 | 259.5 |

TABLE 4
Sitagliptin 47.3 mg mini-tablets (10 mg sitagliptin per mini-tablet = 10
mini-tablets to give 100 mg of active ingredient = 5 mini-tablets to
give 50 mg of active ingredient).

| | F19 | F20 | F21 | F22 | F23 | F24 |
|---|---|---|---|---|---|---|
| Ingredients of MR MiniCore | | | | | | |
| Sitagliptin | 10 | 10 | 10 | 5 | 5 | 5 |
| Microcrystalline cellulose | 20 | 20 | 20 | 10 | 10 | 10 |
| Calcium phosphate | 4.5 | 4.5 | 4.5 | 2.25 | 2.25 | 2.25 |
| HPMC 100 lv | | | | | | 2 |
| HPMC K4M | 3 | 3 | 3 | 2 | 2 | |
| HPMC 15M | | | | | | 3 |
| HPMC K100M | 6 | 6 | 6 | 3 | 3 | |
| Eudragit L100/55 | 0.09 | | 0.09 | 0.045 | | 0.045 |
| Eudragit RL100 | | | | | 0.0225 | |
| Eudragit RS100 | | | | | 0.0225 | |
| Eudragit L100 | | 0.045 | | | | |
| Eudragit S100 | | 0.045 | | | | |
| Shellac | | | | | 0.0225 | |
| Talc | 0.45 | 0.45 | 0.45 | 0.225 | 0.225 | 0.225 |
| Mg stearate | 0.26 | 0.26 | 0.26 | 0.13 | 0.13 | 0.13 |
| | 44.3 | 44.3 | 44.3 | 22.65 | 22.67 | 22.65 |
| Ingredients of IR core | | | | | | |
| Sitagliptin | | | | 5 | 5 | 5 |
| Microcrystalline cellulose | | | | 5 | 5 | 5 |
| Calcium phosphate | | | | 2.25 | 2.25 | 2.25 |
| Crosslinked PVP | | | | 2.5 | 2.5 | 2.5 |
| AcDisol | | | | 2 | 2 | 2 |
| Talc | | | | 0.27 | 0.27 | 0.27 |
| Mg stearate | | | | 0.13 | 0.13 | 0.13 |
| Total | | | | 17.15 | 17.15 | 17.15 |
| Film-coating ingredients | | | | | | |
| Talc | 0.861 | 0.861 | 0.861 | 0.861 | 0.861 | 0.861 |
| Eudragit L100/55 | 1.694 | | 1.694 | 1.694 | | 1.694 |
| Eudragit L100 | | 0.847 | | | | |
| Eudragit S100 | | 0.847 | | | | |
| Shellac | | | | | 1.694 | |
| Ethylcellulose | | | 2.7 | | | 2.7 |
| Titanium dioxide | 0.290 | 0.290 | 0.290 | 0.290 | 0.290 | 0.290 |
| Triethyl citrate | 0.155 | 0.155 | 0.155 | 0.155 | 0.155 | 0.155 |
| Total | 3.0 | 3.0 | 5.7 | 3.0 | 3.0 | 5.7 |
| Grand total | 47.3 | 47.3 | 50.0 | 42.8 | 42.82 | 45.5 |

TABLE 5
Sitagliptin 47.3 mg mini-tablets (5 mg sitagliptin per mini-tablet = 5
mini-tablets to give 25 mg of active ingredient).

| | F25 | F26 | F27 | F28 | F29 | F30 |
|---|---|---|---|---|---|---|
| Ingredients of MR MiniCore | | | | | | |
| Sitagliptin | 5 | 5 | 5 | 2.5 | 2.5 | 2.5 |
| Microcrystalline cellulose | 24 | 24 | 24 | 18 | 18 | 18 |
| Calcium phosphate | 9.5 | 9.5 | 9.5 | 4.75 | 4.75 | 4.75 |
| HPMC K4M | 3 | 3 | 3 | 1 | 1 | 1 |
| HPMC K100M | 2 | 2 | 2 | 2 | 2 | 2 |
| Eudragit L100/55 | 0.09 | | 0.09 | 0.045 | | 0.045 |
| Eudragit L100 | | 0.045 | | | 0.0225 | |
| Eudragit S100 | | 0.045 | | | 0.0225 | |
| Talc | 0.45 | 0.45 | 0.45 | 0.225 | 0.225 | 0.225 |
| Mg stearate | 0.26 | 0.26 | 0.26 | 0.13 | 0.13 | 0.13 |
| | 44.3 | 44.3 | 44.3 | 28.65 | 28.65 | 28.65 |
| Ingredients of IR core | | | | | | |
| Sitagliptin | | | | 2.5 | 2.5 | 2.5 |
| Microcrystalline cellulose | | | | 6 | 6 | 6 |
| Calcium phosphate | | | | 4.75 | 4.75 | 4.75 |
| Crosslinked PVP | | | | 2.5 | 2.5 | 2.5 |
| AcDisol | | | | 2 | 2 | 2 |
| Talc | | | | 0.27 | 0.27 | 0.27 |
| Mg stearate | | | | 0.13 | 0.13 | 0.13 |
| Total | | | | 18.15 | 18.15 | 18.15 |
| Film-coating ingredients | | | | | | |
| Talc | 0.861 | 0.861 | 0.861 | 0.861 | 0.861 | 0.861 |
| Eudragit L100/55 | 1.694 | | 1.694 | 1.694 | | 1.694 |
| Eudragit L100 | | 0.847 | | | 0.847 | |
| Eudragit S100 | | 0.847 | | | 0.847 | |
| Ethylcellulose | | | 2.7 | | | 2.7 |
| Titanium dioxide | 0.290 | 0.290 | 0.290 | 0.290 | 0.290 | 0.290 |
| Triethyl citrate | 0.155 | 0.155 | 0.155 | 0.155 | 0.155 | 0.155 |
| Total | 3.0 | 3.0 | 5.7 | 3.0 | 3.0 | 5.7 |
| Grand total | 47.3 | 47.3 | 50.0 | 49.8 | 49.8 | 52.5 |

TABLE 6
Sitagliptin 100 mg tablets

| | F31 | F32 | F33 | F34 | F35 | F36 | F37 | F38 | F39 |
|---|---|---|---|---|---|---|---|---|---|
| Ingr. of MR Core | | | | | | | | | |
| Saxagliptin | | | | | | | 5 | | |
| Linagliptin | | | | | | | | 5 | |
| Vildagliptin | | | | | | | | | 50 |
| Sitagliptin | 100 | 100 | 100 | 50 | 50 | 50 | | | |
| Calcium phosphate | 190 | 190 | 190 | 90 | 90 | 90 | 190 | 190 | 190 |
| Microcrystalline | 45 | 45 | 45 | 35 | 35 | 35 | 45 | 45 | 45 |
| cellulose | | | | | | | | | |
| HPMC 100 lv | | | | | 20 | | | | |
| HPMC K4M | 40 | 40 | 40 | 15 | | 20 | 40 | 40 | 40 |
| HPMC 15M | | | | | 25 | | | | |
| HPMC K100M | 50 | 50 | 50 | 30 | | 25 | 50 | 50 | 50 |
| Eudragit RS100 | 0.45 | | 0.45 | 0.225 | | 0.225 | 0.45 | 0.45 | 0.45 |
| Eudragit RL100 | 0.45 | | 0.45 | 0.225 | | 0.225 | 0.45 | 0.45 | 0.45 |
| Eudragit L100 | | 0.45 | | | 0.225 | | | | |
| Eudragit S100 | | 0.45 | | | 0.225 | | | | |
| Talc | 4.5 | 4.5 | 4.5 | 2.55 | 2.55 | 2.55 | 4.5 | 4.5 | 4.5 |
| Mg stearate | 2.6 | 2.6 | 2.6 | 1.5 | 1.5 | 1.5 | 2.6 | 2.6 | 2.6 |
| | 433 | 433 | 433 | 224.5 | 224.5 | 224.5 | 338 | 338 | 383 |
| Ingredients of IR core | | | | | | | | | |
| Sitagliptin phosphate | | | | 50 | 50 | 50 | | | |
| anhydrous | | | | | | | | | |
| Calcium phosphate | | | | 50 | 50 | 50 | | | |
| Microcrystalline | | | | 11.5 | 11.5 | 11.5 | | | |
| cellulose | | | | | | | | | |
| Crosslinked PVP | | | | 25 | 25 | 25 | | | |
| AcDisol | | | | 20 | 20 | 20 | | | |
| Talc | | | | 2.7 | 2.7 | 2.7 | | | |
| Mg stearate | | | | 1.3 | 1.3 | 1.3 | | | |
| Total | | | | 160.5 | 160.5 | 160.5 | | | |
| Film-coating ingr. | | | | | | | | | |
| Talc | 8 | 8 | 5 | 5 | 5 | 7 | 8 | 8 | 8 |
| HPMC E5 Premium | 16 | 16 | | 22 | 22 | | 16 | 16 | 16 |
| Ethylcellulose | | | 19 | | | 19 | | | |
| Titanium dioxide | 1 | 1 | 1 | 1 | 1 | 1.5 | 1 | 1 | 1 |
| Triethyl citrate | 2 | 2 | 2 | 2 | 2 | 2.5 | 2 | 2 | 2 |
| Total | 27 | 27 | 47 | 30 | 30 | 30 | 27 | 27 | 27 |
| Grand total | 460 | 460 | 460 | 415 | 415 | 415 | 365 | 365 | 410 |

1. A controlled-release solid oral pharmaceutical composition comprising a core comprising a dipeptidyl peptidase IV inhibitor and an outer coating of said core, wherein:
a) the core comprises:
(i) a monolithic matrix containing sitagliptin, at least one hydroxypropyl methylcellulose having a viscosity ranging between 3 and 5000 mPa·s 2% in H₂O at 20° C., at least one hydroxypropyl methylcellulose having a viscosity ranging between 13500 and 280000 mPa·s 2% in H₂O at 20° C., at least one or more methacrylic polymers/copolymers and/or shellac, cellulose acetate phthalate, hydroxypropyl methylcellulose succinate acetate, or
(ii) a monolithic matrix as defined above adjacent to an immediate-release layer comprising a dipeptidyl peptidase IV inhibitor;
b) the outer coating comprises a layer comprising hydroxypropyl methylcellulose and/or ethylcellulose or a gastroresistant layer or a layer comprising ethylcellulose which in turn is coated with gastroresistant polymers.

2. A composition as claimed in claim 1 wherein the core comprises a monolithic matrix as defined in claim 1, point (i).

3. A composition as claimed in claim 1 wherein the core comprises a monolithic matrix as defined in claim 1, adjacent to an immediate-release layer comprising the dipeptidyl peptidase IV inhibitor.

4. A composition as claimed in claim 1 wherein the outer coating comprises a layer comprising ethylcellulose.

5. A composition as claimed in claim 1 wherein the outer coating comprises a layer comprising ethylcellulose coated with gastroresistant polymers.

6. A composition as claimed in claim 1 wherein the outer coating comprises a gastroresistant layer.

7. A composition as claimed in claim 1 wherein the acrylic/methacrylic polymers or copolymers are selected from pH-independent methacrylic ester copolymers, pH-independent ammonium alkyl methacrylate copolymers; amino alkyl methacrylate copolymers soluble up to pH 5.0, methacrylic acid copolymers soluble at pH≥5.5, methacrylic acid copolymers soluble at pH 6.0-7.0; and pH-dependent methacrylic acid copolymers soluble at pH≥7.0.

8. A composition as claimed in claim 1 wherein the monolithic matrix comprises shellac.

9. A composition as claimed in claim 1 wherein the gastroresistant coating comprises pH-dependent methacrylic acid copolymers soluble at pH≥5.5; pH-dependent methacrylic acid copolymers soluble at pH 6.0-7.0; pH-dependent methacrylic acid copolymers soluble at pH≥7.0; shellac; cellulose acetate phthalate or cellulose succinate.

10. A composition as claimed in claim 1 wherein the hydroxypropyl methylcellulose having a viscosity ranging between 3 and 5000 mPa·s 2% in H₂O at 20° C. constitutes 1 to 20% of the weight of the core, the hydroxypropyl methylcellulose having a viscosity ranging between 13500 and 280000 mPa·s 2% in H₂O at 20° C. constitutes 1 to 20% of the weight of the core, and the methacrylic polymer/copolymer constitutes 0.1 to 2% of the weight of the core.

11. A composition as claimed in claim 1 wherein ethylcellulose is present in percentages ranging from 1 to 20% of the weight of the core.

12. A composition as claimed in claim 1 wherein the dipeptidyl peptidase IV (DPP-4) inhibitor is sitagliptin, vildagliptin, saxagliptin or linagliptin.

13. A composition as claimed in claim 1, wherein the dipeptidyl peptidase IV (DPP-4) inhibitor is sitagliptin.
